(19) United States
(12) Patent Application Publication
(10) Pub. No.: US 2026/0157655 A1
(43) Pub. Date: Jun. 11, 2026

(54) HAND THERAPY DEVICE

(71) Applicant: MindMaze Group SA, Lausanne (CH)

(72) Inventors: Omar AHMAD, Baltimore, MD (US); Kevin MURPHY, Media, PA (US); Joshua FUNAMURA, Oakland, CA (US); Justin BECHSTEIN, Philadelphia, PA (US); Jason ZERWECK, Media, PA (US); Christopher MARKS, Philadelphia, PA (US)

(21) Appl. No.: 18/702,841

(22) PCT Filed: Oct. 20, 2022
(86) PCT No.: PCT/IB2022/060112

§ 371 Date: Apr. 19, 2024

Related U.S. Application Data
(60) Provisional application No. 63/257,815, filed on Oct. 20, 2021.

(51) Int. Cl. A61B 5/11

(52) U.S. Cl. CPC A61B 5/1114; A61B 5/1124; A61B 2505/09

(57) ABSTRACT

A finger sensor stage assembly connected to a finger cup and detecting motion in at least two degrees of freedom caused by forces generated by a finger includes a first stage block coupled to the finger cup; a second stage block; at least one first flexure connected to the first and second stage blocks and restricting relative movement of the first and second stage blocks to a first degree of freedom; a third stage block; at least one second flexure connected to the second and third stage blocks and restricting relative movement of the second and third stage blocks to a second degree of freedom different from the first; and one or more non-contact position sensors to detect relative movement of the first and second stage blocks in the first degree of freedom and relative movement of the second and third stage blocks in the second degree of freedom.

### BACKGROUND

Embodiments described herein relate generally to a hand therapy device, and more particularly, to a hand therapy device that can consistently and reliably measure small forces exhibited by isolated fingers in multiple degrees of freedom.

Measuring dexterity in individual fingers can be an important component in testing and recovery training of victims of certain neurological conditions, such as a stroke. Strength and dexterity are independent of one another, and while numerous devices and therapies can measure strength, which is more indicative of muscular capabilities than brain control, dexterity measurements and training are not as prevalent. Other conditions for which dexterity testing and training can be beneficial include Parkinson's disease, carpel-tunnel syndrome, and arthritis, to name but a few.

Existing devices suffer from numerous drawbacks. Many of the devices rely on the use of strain gauges or other types of solid-state sensors, which can lack precision and reliability to measure small forces, are subject to breakage, and may have issues caused by measurement drift (e.g., from resistance changes caused by temperature fluctuation). Such devices also tend to have high price tags, often due to the types of sensors utilized.

It is therefore desirable to provide a hand therapy device capable of isolating fingers to measure small forces (e.g., on the order of milli-Newtons (mN)) related to dexterity movements that may be otherwise imperceptible to the human eye. It is further desirable for such a device to be able to obtain such measurements stably and consistently for a plurality of degrees of freedom. It is further desirable for such a device to enable adjustment for accommodating a neutral rest position for hands of different sizes and shapes, and to be able to repeat a particular user's exact rest position for accurate comparisons with past measurements. It is further desirable to be able to use such a device with training software programming for improving dexterity movements.

### BRIEF SUMMARY

Briefly stated, an embodiment comprises a finger sensor stage assembly connected to a finger cup configured to receive at least a portion of a finger, the finger sensor stage assembly being configured to detect motion in at least two degrees of freedom caused by forces generated by the finger on the finger cup, the finger sensor stage assembly including a first stage block coupled to the finger cup; a second stage block; at least one first flexure connected to the first stage block and to the second stage block and restricting relative movement of the first stage block and the second stage block to a first degree of freedom; a third stage block; at least one second flexure connected to the second stage block and to the third stage block and restricting relative movement of the second stage block and the third stage block to a second degree of freedom different from the first degree of freedom; and one or more non-contact position sensors configured to detect the relative movement of the first stage block and the second stage block in the first degree of freedom and the relative movement of the second stage block and the third stage block in the second degree of freedom.

In one aspect, the finger sensor stage assembly further includes a fourth stage block; and at least one third flexure connected to the third stage block and to the fourth stage block and restricting relative movement of the third stage block and the fourth stage block to a third degree of freedom different from the first and second degrees of freedom, wherein the one or more non-contact position sensors are further configured to detect the relative movement of the third stage block and the fourth stage block in the third degree of freedom.

In another aspect, the finger sensor stage assembly further includes a fifth stage block; and at least one fourth flexure connected to the fourth stage block and to the fifth stage block and restricting relative movement of the fourth stage block and the fifth stage block to a fourth degree of freedom different from the first, second, and third degrees of freedom, wherein the one or more non-contact position sensors are further configured to detect the relative movement of the fourth stage block and the fifth stage block in the fourth degree of freedom.

In another aspect, the finger sensor stage assembly further includes a sixth stage block; and at least one fifth flexure connected to the fifth stage block and to the sixth stage block and restricting relative movement of the fifth stage block and the sixth stage block to a fifth degree of freedom different from the first, second, third, and fourth degrees of freedom, wherein the one or more non-contact position sensors are further configured to detect the relative movement of the fifth stage block and the sixth stage block in the fifth degree of freedom.

In another aspect, the one or more non-contact position sensors include a first magnet supported by the first stage block and a corresponding first Hall effect sensor supported by the third stage block, the first magnet and first Hall effect sensor being configured to detect the relative movement of the first stage block and the second stage block in the first degree of freedom and the relative movement of the second stage block and the third stage block in the second degree of freedom, a second magnet supported by the third stage block and a corresponding second Hall effect sensor supported by the fourth stage block, the second magnet and the second Hall effect sensor being configured to detect the relative movement of the third stage block and the fourth stage block in the third degree of freedom, and a third magnet supported by the fourth stage block and a corresponding third Hall effect sensor supported by the sixth stage block, the third magnet and the third Hall effect sensor being configured to detect the relative movement of the fourth stage block and the fifth stage block in the fourth degree of freedom and the relative movement of the fifth stage block and the sixth stage block in the fifth degree of freedom.

In another aspect, the first and second degrees of freedom are rotational and the third, fourth, and fifth degrees of freedom are translational.

In another aspect, each of the at least one first flexure and the at least one second flexure is a spring plate.

In another aspect, the at least one first flexure includes two pairs of parallel, spaced-apart spring plates and the at least one second flexure includes two pairs of parallel, spaced-apart spring plates.

In another aspect, each of the one or more non-contact position sensors comprises a magnet and a corresponding Hall effect sensor.

In another aspect, the one or more non-contact position sensors comprises a single non-contact position sensor having the magnet supported by the first stage block and the Hall effect sensor supported by the third stage block.

In another aspect, the finger sensor assembly further includes a finger hub printed circuit board communicatively connected to the one or more non-contact position sensors for receiving measurement data from the one or more non-contact position sensors.

In another aspect, the finger hub printed circuit board includes a communication port configured to transmit the measurement data received from the one or more sensors.

Another embodiment comprises a hand therapy device including five finger sensor stage assemblies described above.

Another embodiment comprises a hand therapy device including a hand module assembly including a housing, and five finger sensor assemblies arranged at least partially within the housing, each of the finger sensor assemblies including: a finger cup configured to receive at least a portion of a finger, a finger sensor stage assembly connected to the finger cup and including one or more non-contact position sensors configured to detect motion in at least two degrees of freedom caused by forces generated by the finger on the finger cup, an adjustment slide rail, and an adjustment bracket connected to at least one of the finger cup or the finger sensor stage assembly and selectively movable and lockable relative to the adjustment slide rail in at least one translational direction and rotatably, wherein a position of one of the finger cups relative to the other finger cups is adjustable by movement of the corresponding adjustment bracket relative to the corresponding adjustment slide rail.

In one aspect, the hand therapy device further includes a base, the hand module assembly being supported by the base.

In another aspect, the hand therapy device further includes an arm connecting the base to the hand module assembly.

In another aspect, the arm is rotatable with respect to the base.

In another aspect, the hand therapy device further includes a rotating collar supported by the base and connected to the arm; and a wrist knob attached to the rotating collar, the wrist knob including a rotatable dial such that rotating the rotatable dial in a first direction expands the rotating collar to allow the rotating collar to rotate relative to the base and adjust a position of the arm relative to the base, and rotating the rotatable dial in a second direction opposite to the first direction tightens the rotating collar to prevent rotation of the rotating collar relative to the base and lock the arm in position relative to the base.

In another aspect, the hand therapy device further includes an arm rest supported by the base.

In another aspect, the hand module assembly further includes a chassis selectively movable and lockable relative to the housing, the chassis containing at least four of the finger sensor assemblies.

In another aspect, the housing includes an indicator slot and the chassis includes a cooperating indicator pin extending within the indicator slot, the indicator pin allowing selective translational movement of the chassis relative to the housing in a direction parallel to a longitudinal direction of the indicator slot.

In another aspect, the hand module assembly further includes a finger knob rotatably mounted on the housing, wherein rotating the finger knob in a first direction allows the chassis to move relative to the housing as guided by the indicator pin within the indicator slot, and rotating the finger knob in a second, opposite direction locks the chassis to the housing.

In another aspect, the hand module assembly further includes a microcontroller unit disposed within the housing and communicatively connected to each of the one or more sensors from the five finger sensor assemblies.

In another aspect, the microcontroller unit includes at least one communication module for sending data related to the one or more sensors.

In another aspect, the at least one communication module includes a USB port.

In another aspect, each of the finger sensor assemblies further includes a brake housing attached to the adjustment bracket and selectively movable with respect to the adjustment slide rail, and an adjustment lever selectively pivotable relative to the brake housing, wherein when the adjustment lever is in a first position relative to the brake housing, the brake housing and the adjustment bracket are movable relative to the adjustment slide rail, and when the adjustment lever is in a second position relative to the brake housing, the brake housing and the adjustment bracket are prevented from moving relative to the adjustment slide rail.

In another aspect, the adjustment lever is biased toward the second position by a spring plunger positioned between the brake housing and the adjustment lever.

In another aspect, each finger cup includes a finger cup base and a finger cup top that is selectively movable toward or away from the finger cup base.

In another aspect, each finger cup further includes a finger cup lever and a lever support, the lever support being connected to the finger cup top for movement therewith, the finger cup lever being selectively pivotable with respect to the lever support, wherein when the finger cup lever is in a first position relative to the lever support, the lever support and finger cup top are free to move relative to the finger cup base, and when the finger cup lever is in a second position relative to the lever support, the lever support and finger cup top are prevented from moving relative to the finger cup base.

Another embodiment comprises a method of operating a hand therapy device, the hand therapy device including a microcontroller unit and a hand module assembly that includes a housing and five finger sensor assemblies arranged at least partially within the housing, each of the finger sensor assemblies including a finger cup configured to receive at least a portion of a finger and a finger sensor stage assembly connected to the finger cup and including one or more non-contact position sensors configured to detect motion in at least two degrees of freedom caused by forces generated by the finger on the finger cup, the method including adjusting a position of at least one of the finger sensor assemblies relative to the other finger sensor assemblies to establish a neutral rest position for a hand of a user to be received by the hand module assembly; recording the positions of the finger sensor assemblies after establishing the neutral rest position for the hand of the user; receiving, by the finger cups of the finger sensor assemblies, corresponding fingers of the hand of the user; and obtaining, by the microcontroller unit, data from the one or more non-contact position sensors of at least one of the finger stage assemblies, the data representing movement of the respective at least one finger stage assembly caused by forces generated by the respective finger.

In another aspect, the method further includes resetting, by the microcontroller unit after establishing the neutral rest position for the hand of the user, the one or more non-contact position sensors in the finger stage assemblies to account for a direction of gravity relative to the finger stage assemblies.

In another aspect, the hand therapy device further includes an arm rest and a base supporting the arm rest and the hand module assembly, the method further including adjusting a position of the hand module assembly relative to the arm rest to establish the neutral rest position for the hand of the user; and recording the position of the hand module assembly after establishing the neutral rest position for the hand of the user.

In another aspect, a size of the finger cups of the finger sensor assemblies is adjustable, the method further including adjusting the size of at least one of the finger cups; and recording the sizes of the finger cups after establishing the neutral rest position for the hand of the user.

In another aspect, the hand module assembly further includes a chassis containing at least four of the finger sensor assemblies, the method further including adjusting a position of the chassis relative to the housing to establish the neutral rest position for the hand of the user; and recording the position of the chassis after establishing the neutral rest position for the hand of the user.

### BRIEF DESCRIPTION OF THE SEVERAL VIEWS OF THE DRAWINGS

The following detailed description of preferred embodiments will be better understood when read in conjunction with the appended drawings. For the purpose of illustration, there are shown in the drawings embodiments which are presently preferred. It should be understood, however, that the invention is not limited to the precise arrangements and instrumentalities shown.

In the drawings:

FIG. 1 is a left side perspective view of a hand therapy device in accordance with an example embodiment of the present invention;

FIG. 2 is front side perspective view of the hand therapy device of FIG. 1;

FIG. 3 is a right side perspective view of the hand therapy device of FIG. 1;

FIG. 4 is a left side perspective view of a mechanism for adjusting relative positioning of a hand module assembly and an arm rest of the hand therapy device of FIG. 1;

FIG. 5 is a right side perspective view of a finger sensor mount and thumb sensor mount of the hand therapy device of FIG. 1;

FIG. 6 is a bottom and left side perspective view of a finger sensor assembly of the hand therapy device of FIG. 1;

FIG. 7 is a bottom side perspective view of a finger sensor stage assembly of the finger sensor assembly of FIG. 6;

FIG. 8 is a top side perspective view of a position sensor and flex printed circuit of the finger sensor stage assembly of FIG. 7;

FIG. 9 is a bottom side perspective view of a stage block and magnet cooperating with the position sensor of FIG. 8;

FIG. 10 is a bottom and right side perspective exploded view of stage blocks utilized in the finger sensor stage assembly of FIG. 7; and

FIG. 11 is a schematic block circuit diagram for use with the hand therapy device of FIG. 1.

### DETAILED DESCRIPTION

Certain terminology is used in the following description for convenience only and is not limiting. For example, words such as “right,” “left,” “lower,” “upper,” “front,” “back,” “top,” “bottom,” “over,” “under,” and the like, if any, are used for descriptive purposes in the drawings to which reference is made, and not necessarily for describing permanent relative positions. It is understood that the terms so used are interchangeable under appropriate circumstances such that the embodiments of the invention described herein are, for example, capable of operation in other orientations than those illustrated or otherwise described herein.

The words “inwardly” and “outwardly” refer to directions toward and away from, respectively, the geometric center of the device and designated parts thereof. The terminology includes the above-listed words, derivatives thereof, and words of similar import. Additionally, the words “a” and “an”, as used in the claims and in the corresponding portions of the specification, mean “at least one.”

It should also be understood that the terms “about,” “approximately,” “generally,” “substantially,” and like terms, used herein when referring to a dimension or characteristic of a component, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, or the like), would not vary the least significant digit.

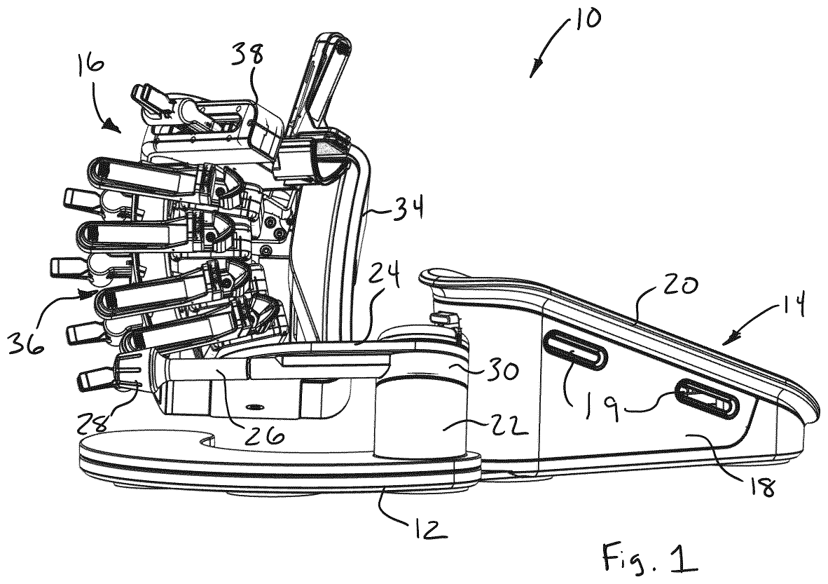

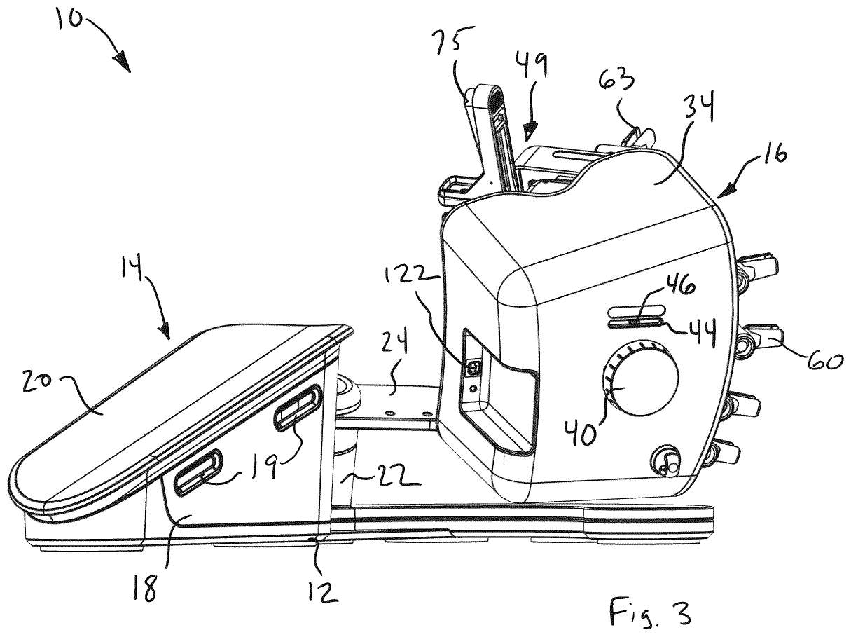

Referring to FIGS. 1-3, there is shown an example embodiment of a hand therapy device, generally designated by reference numeral 10. The hand therapy device 10 may include a base 12 configured to support an arm rest 14 and an associated hand module assembly 16. However, multiple separate bases (not shown) may be used, for example, one for each of the arm rest 14 and the hand module assembly 16 without departing from the scope of the invention. The base 12 preferably allows the hand therapy device 10 to be movably placed on a table top, desk top, or other similar support surface (not shown) for use by a patient or other user. Alternatively, the arm rest 14 and/or the hand module assembly 16 may be secured to or otherwise permanently integrated with a self-supporting, floor-mounted structure (not shown).

The hand therapy device 10 shown in FIGS. 1-3 is configured to receive a left hand of a user. One skilled in the art recognizes that a hand therapy device configured to receive a right hand would include the same features but with some of the components reversed, for example, forming a mirror image of the hand therapy device 10 of FIGS. 1-3. However, it is also possible to construct a hand therapy device that can be reversible, such as by pivoting, rotating, and/or removing and replacing various components.

The arm rest 14 may include a support body 18 disposed on the base 12. The support body 18 shown in FIGS. 1-3 exhibits a top inclined surface supporting a cradle 20 configured to receive an arm of the user when the hand therapy device 10 is in use. At least a portion of the cradle 20 may be padded for the user's comfort. The support body 18 may include a plurality of pairs of strap slots 19 formed in sidewalls thereof. Straps (not shown) may be fed through the strap slots 19 and used to secure a user's arm to the cradle 20 for maintaining a steady position during use of the hand therapy device 10. The cradle 20 may be pivotally connected or completely detachable from the support body 18 to provide access to an interior of the support body 18 to make feeding of the straps through the strap slots 19 more convenient. The support body 18 may also be used for storage of the straps or other loose components affiliated with the hand therapy device 10. For example, cables for connecting the hand therapy device 10 to an external computer (not shown) or the like may be stored in the support body 18.

In addition, a height of the cradle 20 and/or the support body 18 may be adjustable with respect to the base 12 to permit the user, regardless of hand or arm size, to attain a comfortable resting position of the hand and arm. In addition to height adjustment, the incline of the support body 18 (or in some instances, of the cradle 20) may also be adjustable. For example, the support body 18 may be movable between a position such as that shown in FIGS. 1-3 and a position (not shown) where the top surface supporting the cradle 20 is substantially flat and parallel to the base 12.

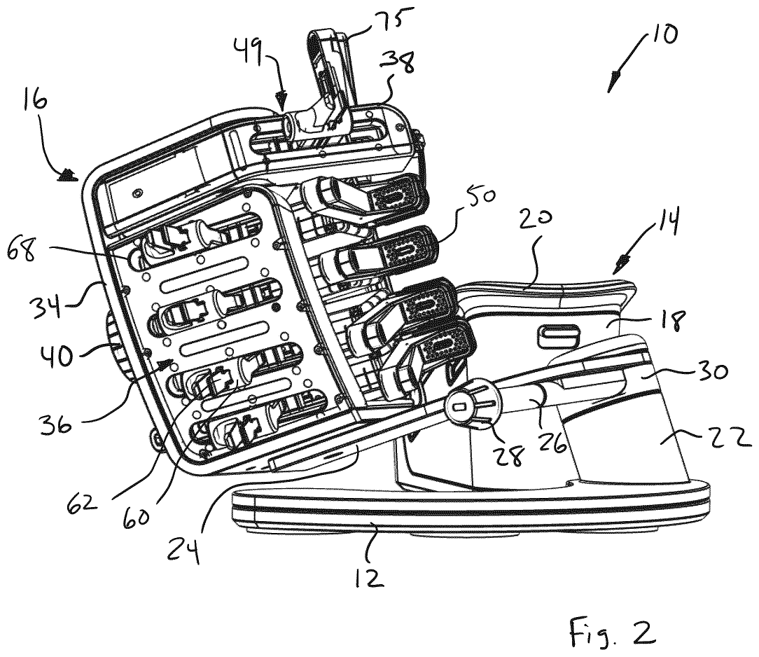

The hand module assembly 16 may be mounted at an angle with respect to a longitudinal axis of the arm rest 14 so that the user's hand can be placed in a comfortable, neutral position while the user's arm is supported by the arm rest 14. For example, the base 12 may include a column 22 extending generally vertically therefrom. In FIG. 2, in particular, the column 22 is shown at a non-perpendicular angle with respect to the base 12, although the column 22 may extend, or be adjusted to extend, substantially perpendicularly to the base 12.

An arm 24 may have one end thereof coupled to the column 22. The hand module assembly 16 may be attached to an opposing end of the rotating arm 24. In some embodiments, the hand module assembly 16 is configured to accept a standard tripod mount (not shown), e.g., a ¼-20 screw and one or more depressible pins. In this way, the hand module assembly 16 may be decoupled from the arm 24 for use with other equipment where use of the complete hand therapy device 10 structure is impractical (e.g., by a bedside or the like). The arm 24 is shown in FIGS. 1-3 having a general L-type shape, but other shapes are possible, and the arm 24 may be formed by multiple components that are rigidly or movably joined together. The arm 24 may also be integrally formed with a portion of the hand module assembly 16 rather than being simply attached thereto.

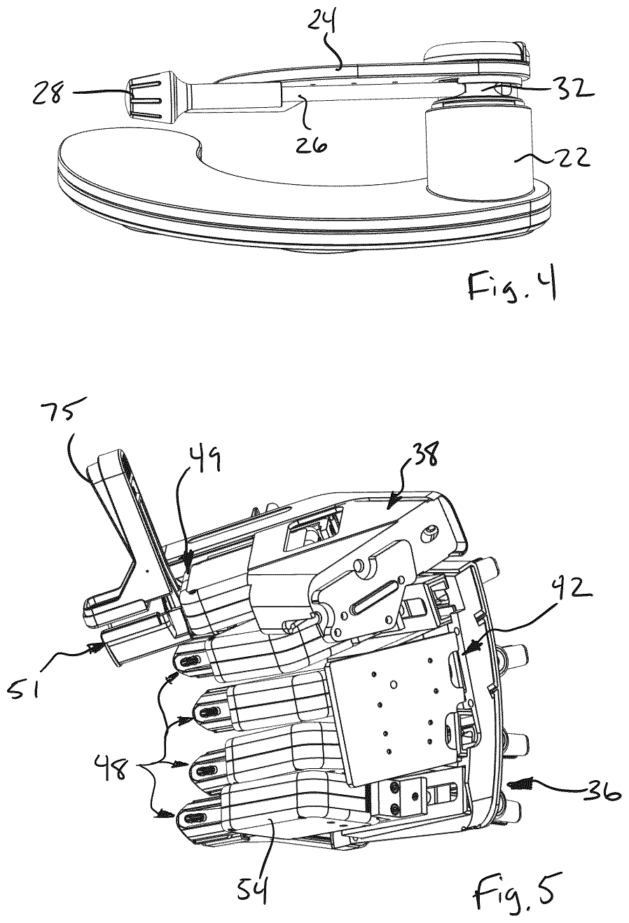

The hand module assembly 16 is preferably pivotable with respect to the arm rest 14 to allow for adjustments to accommodate different users in respective comfortable, neutral positions. Rotation may be provided by pivotal attachment of the arm 24 to the column 22. A wrist knob 26 may connect to the arm 24 and column 22 to facilitate the rotational movement. A free end of the wrist knob 26 may include a dial 28 utilized to rotate the wrist knob about its longitudinal axis interesting the column 22. As seen in FIG. 4, the wrist knob 26 may extend through an arm lock cover 30 (FIGS. 1-2) and attach to a rotating collar 32. The rotating collar 32 may be coupled to the arm 24 such that rotation of the collar 32 causes pivoting of the arm 24 about the column 22. Turning the dial 28 in a counter-clockwise direction may cause a threaded end (not shown) of the wrist knob 26 to allow circumferential expansion of the rotating collar 32, permitting pivoting of the arm 24. Turning the dial 28 in the opposite direction can cause the threaded end of the wrist knob 26 to contract the circumference of the rotating collar 32 about a stationary pillar (not shown), resulting in a frictional locking of the rotating collar 32, preventing movement of the arm 24 and locking the hand module assembly 16 in a desired location relative to the arm rest 14. However, other methods and mechanisms for adjusting the relative position of the hand module assembly 16 and the arm rest 14 can be used as well within the scope of the invention.

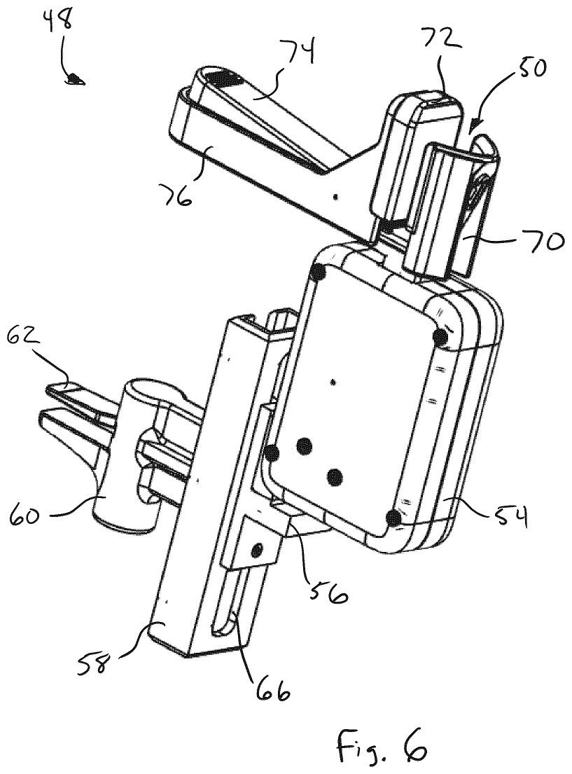

Referring again to FIGS. 1-3, the hand module assembly 16 may include a housing 34 at least partially enclosing a finger sensor mount 36 and a thumb sensor mount 38 (shown together in FIG. 5), as well as operating electronics, such as those described in further detail below. The housing 34 may include any attachment hardware for coupling the hand module assembly 16 to the arm 24. The thumb sensor mount 38 may be directly connected to the housing 34 for movement therewith, as may the finger sensor mount 36. However, in the embodiment shown in FIGS. 1-3, the finger sensor mount 36 may be movable in at least one direction relative to the housing 34 to allow for adjustments to accommodate differing hand sizes and finger spacings. In some other embodiments, the thumb sensor mount 38 may also be movable relative to the housing 34.

A finger knob 40 may be accessible on an exterior side of the housing 34 and include a pin (not shown) that extends through a slot (not shown) in the housing 34 and connects to a chassis 42 of the finger sensor mount 36. When the finger knob 40 is tightened, the chassis 42 may be held in place relative to the housing 34. When the finger knob 40 is loosened, the chassis 42 can be translated in a direction parallel to the indicator slot 44 shown in the housing 34. An indicator pin 46 may be connected to the chassis 42 for movement therewith (or formed as part of the chassis) to extend through the indicator slot 44 for indicating a position of the chassis 42 within the housing 34. Once the finger sensor mount 36 is in the desired location, the finger knob 40 can be re-tightened to hold the chassis 42 in place relative to the housing 34.

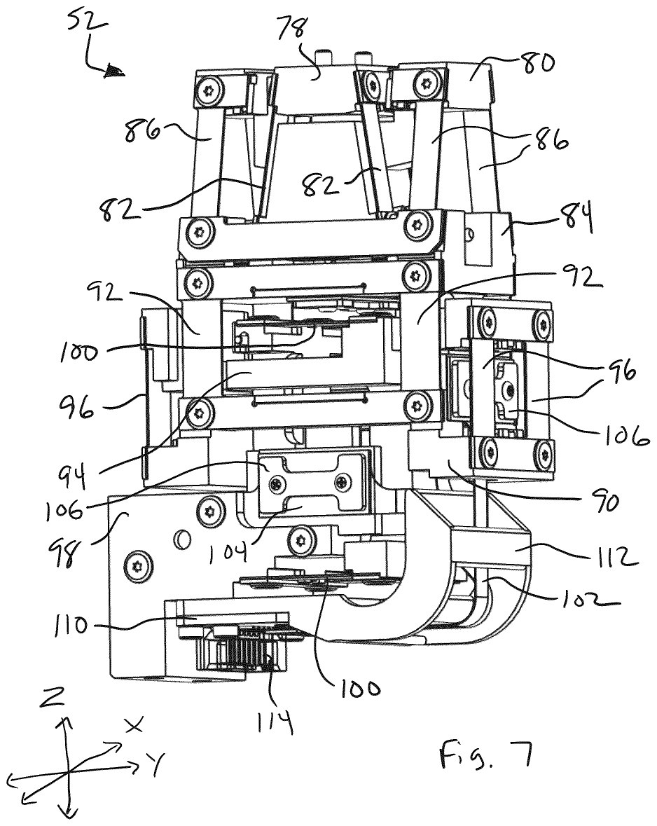

The finger sensor mount 36 may contain four finger sensor assemblies 48. An exemplary finger sensor assembly 48 is shown in FIG. 6. The finger sensor assembly 48 may include a finger cup 50 configured to receive at least a portion of a finger during use of the hand therapy device 10. The finger cup 50 may be operably attached to a finger sensor stage assembly 52 (see e.g., FIG. 7), which is preferably contained within a sensor enclosure 54. The sensor enclosure 54 may be connected to an adjustment bracket 56 at least partially received within an adjustment slide rail 58. The adjustment bracket 56 (and therefore the sensor enclosure 54 and finger cup 50) is preferably selectively movable with respect to the adjustment slide rail 58. For example, a brake housing 60 may extend from a side of the adjustment slide rail 58 opposite to the adjustment bracket 56.

In the example shown in FIG. 6, the brake housing 60 may interact with an adjustment lever 62 coupled to a brake pad (not shown) that may be located within the adjustment slide rail 58. The adjustment lever 62 may be pivotally movable with respect to the brake housing 60. A spring plunger (not shown) may be provided between the adjustment lever 62 and the brake housing 60 to bias the adjustment lever 62 into a position where the brake pad abuts against the adjustment slide rail 58 to inhibit movement of the adjustment bracket 56 with respect to the adjustment slide rail 58. When a user depresses the adjustment lever 62, the brake pad may release from the adjustment slide rail 58 and the adjustment bracket 56 may then be moved. The adjustment slide rail 58 may include a main slot 66, which can allow the adjustment bracket 56 to translate in a direction parallel to the main slot 66. However, it is also contemplated that the adjustment bracket 56 may be sized so as to permit motion relative to the adjustment slide rail 58 in directions transverse to the main slot 66, although a width of the main slot 66 and/or other features of the adjustment slide rail 58 may limit the extent of such movement. Similarly, it is contemplated that the adjustment bracket 56 may be allowed to rotate relative to the adjustment slide rail 58 within the main slot 66. Such rotation may be restricted to about 15°-20°, although other ranges of rotation may be utilized. When the adjustment lever 62 is released, the spring plunger causes the adjustment lever 62 to return to its rest position relative to the brake housing 60, which locks the adjustment bracket 56 in place. The spring plunger may have adjustable stiffness, if desired.

The adjustment slide rail 58 may be attached to the chassis 42 of the finger sensor mount 36 and/or the housing 34. For example, the adjustment slide rails 58 of the respective finger sensor assemblies 48 are attached to the chassis 42, and the respective brake housings 60 and adjustment levers 62 extend out through corresponding access slots 68. Thus, changing the location of the adjustment bracket 56 may be used to reposition the finger cups 50 to accommodate the user's hand in a comfortable position.

The finger cup 50 may be formed partially by a finger cup base 70, which as shown in FIG. 6, may have a rounded V-type shape in plan view, generally corresponding to the contour of the bottom of a finger (e.g., the side opposite the nail) that may be received in the finger cup 50, although other shapes may be used as well. In the example shown in FIG. 6, the finger cup 50 may further include a finger cup top 72 arranged opposite to the finger cup base 70. In this example, the finger cup top 72 is generally flat. Together, the finger cup base 70 and the finger cup top 72 cooperate to capture a finger of the user. For comfort of the user, one or both of the finger cup base 70 and the finger cup top 72 may be padded at least in regions that contact the finger. The finger cup base 70 and finger cup top 72 are shown in the drawings as being separated components, but in some other embodiments the finger cup 50 may be formed of a single, annular cylinder or cone-like shape for receiving the user's finger.

To keep the user's finger in place within the finger cup 50, one or both of the finger cup base 70 and the finger cup top 72 may be movable with respect to the other. In the example of FIG. 6, the finger cup top 72 is capable of translating toward and away from the finger cup base 70. This may be accomplished, for example, by a finger cup lever 74 and lever support 76 that may be attached to the finger cup top 72 for movement therewith relative to the finger cup base 70. The finger cup base 70 may, for example, include an adjustment arm (not shown) that is at least partially received by the lever support 76 and which exhibits a series of teeth (not shown), each representing a translational distance from the finger cup base 70. The finger cup lever 74 may include a catch (not shown) configured to cooperate with the teeth for locking the finger cup top 72 in various positions relative to the finger cup base 70. When the user depresses the finger cup lever 74, the catch releases from the teeth. The finger cup top 72, via the lever support 76, can now translate to a new position relative to the finger cup base 70. Upon release of the finger cup lever 74, a return spring (not shown) may return the finger cup lever 74 to its rest position relative to the lever support 76, causing the catch to engage the nearest adjacent tooth and locking the finger cup top 72. Other adjustment and locking mechanisms for securing a user's finger in the finger cup 50 may be used as well.

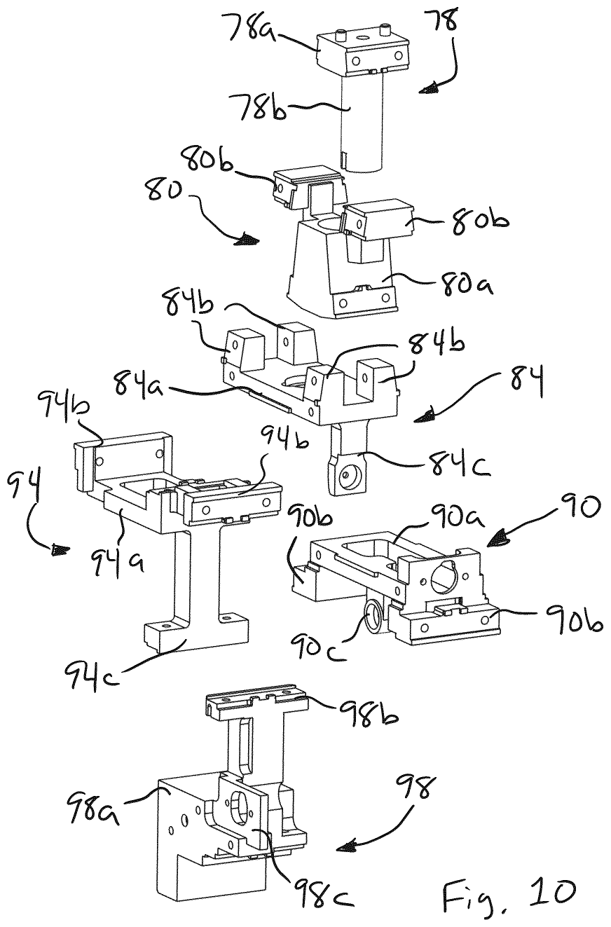

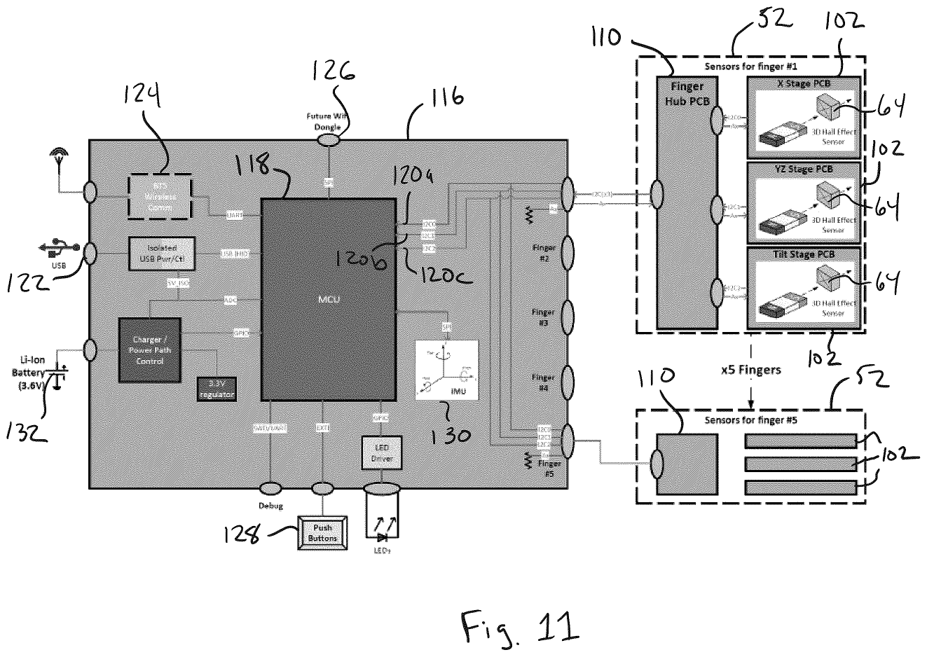

Referring to FIGS. 7-10, an example embodiment of a finger sensor stage assembly 52 will now be described. The finger sensor stage assembly 52 will preferably include the necessary sensors for detecting forces generated by the user's finger in the corresponding finger cup 50 and also preferably isolates motion in each degree of freedom to simplify the mathematical calculations needed to determine the aforementioned forces. In the example shown in FIGS. 7-10, the finger sensor stage assembly 52 obtains measurements in five degrees of freedom, including first, second, and third linear dimensions (e.g., labeled as X, Y, and Z in FIG. 7 for simplicity) and two rotational dimensions (e.g., pitch and roll, wherein pitch refers to rotation about an axis parallel to X and roll refers to rotation about an axis parallel to Y). However, more or fewer degrees of freedom may be used, as necessary.

Preferably, the finger sensor stage assembly 52 uses one or more non-contact position sensors 64 to obtain the necessary readings. In the example shown in FIGS. 7-10, the position sensors are three-dimensional Hall effect sensors, such as the ALS313003DMAG position sensor available from Allegro Microsystems, Inc. of Manchester, New Hampshire. Three position sensors 64 are used in the particular example of FIGS. 7-10: one for detecting pitch and roll rotational movements, one for detecting translational movement in the X-direction, and one for detecting translational movement in the Y-and Z-directions. However, more or fewer position sensors 64 may be used, as needed. The use of non-contact position sensors 64 provides advantages over devices such as strain gauges, which as described above, are subject to breakage, reliability issues, drift, and other similar problems.

In some embodiments, the position sensors 64 may include nonvolatile memory, which may be used to save calibration data, for example. Calibration may be performed using a predetermined weight (e.g., 500 grams) and measuring an amount of resulting movement by the subject position sensor 64. Preferably, the calibration only needs to be performed once, i.e., during manufacture, but in some embodiments, the position sensors 64 may be recalibrated as needed. Position data to be reported out (as described below) may also be saved in nonvolatile memory, although in some embodiments, measurement data may be saved only in a volatile memory.

The finger sensor stage assembly 52 may include a plurality of stage blocks that are used for mounting the position sensors 64 and for attachment of flexures for isolating particular degrees of freedom. The stage blocks are preferably substantially rigid so that force applied by the finger causes relative motion of the stage blocks with respect to one another rather than significant elastic or plastic deformation of a respective stage block. Using the example in FIGS. 7-10, the finger cup 50, and particularly the finger cup base 70, may be attached to a first stage block 78, which in this example includes an attachment head 78a in combination with a generally cylindrical shaft 78b extending therefrom in the Z-direction. In this configuration, the user's finger in the finger cup 50 would extend generally parallel to the Z-direction. The first stage block 78 may be received by a second stage block 80, which in this example includes a rocker base 80a with a rounded edge located opposite (in the Z-direction) to an end that receives the first stage block 78. Two wings 80b may extend from the end of the rocker base 80a opposite the rounded edge and may be spaced apart from one another in the Y-direction. The shaft 78b of the first stage block 78 may be received within the rocker base 80a of the second stage block 80, while the attachment head 78a of the first stage block 78 may be positioned between the two wings 80b of the second stage block 80.

Two pairs of parallel, spaced-apart first flexures 82 may be used to attach the first stage block 78 to the second stage block 80 (one first flexure 82 from each pair is visible in FIG. 7). The first flexures 82 may each be spring plates, although other types of flexures may be used provided that they exhibit the proper stiffness to restrict movement to one degree of freedom. In the example of FIGS. 7-10, each of the first flexures 82 has one end attached to a generally Y-direction facing surface of the rocker base 80a of the second stage block 80 and the other end attached to a generally Y-direction facing surface of the attachment head 78a of the first stage block 78. In this example, a width of the rocker base 80a in the Y-direction is greater than a width of the attachment head 78a, so the first flexures 82 may be inclined toward a center of the finger cup 50. This alignment preferably places the degree of freedom of the first flexures 82 to allow for pitch rotation of the first stage block 78 relative to the second stage block 80 and uses the degrees of constraint of the first flexures 82 to prevent other types of relative motion between the first and second stage blocks 78, 80.

A third stage block 84 may be provided, which may have a planar base 84a and four corner protrusions 84b extending generally in the Z-direction from the planar base 84a. In the example shown in FIGS. 7-10, a portion of the rocker base 80a of the second stage block 80 is received within a volume defined by the corner protrusions 84b of the third stage block 84, although preferably the second stage block 80 does not contact the third stage block 84 in a rest configuration. Rather, the second stage block 80 may be suspended using two pairs of parallel, spaced-apart second flexures 86, each having one end attached to a generally X-direction facing surface of one of the corner protrusions 84b of the third stage block 84 and the other end attached to a generally X-direction facing surface of one of the wings 80b of the second stage block 80.

As before, the second flexures 86 are preferably spring plates, although other types of flexures may be used as well. In this example, a width of each wing 80b in the X-direction is less than a spacing in the X-direction between the surfaces of the corner protrusions 84b to which the second flexures 86 are attached. As a result, the second flexures 86 may be inclined toward a center of the finger cup 50. This alignment preferably places the degree of freedom of the second flexures 86 to allow for roll rotation of the second stage block 80 relative to the third stage block 84 and uses the degrees of constraint of the second flexures 86 to prevent other types of relative motion between the second and third stage blocks 80, 84.

In the example shown in FIGS. 7-10, a position sensor 64 (in this instance, a Hall effect sensor), may be attached to, embedded in, or otherwise supported by the planar base 84a of the third stage block 84 for movement therewith (this particular position sensor 64 is not visible in FIGS. 7-10). A magnet 88 (not visible in FIGS. 7-10) may be attached to, embedded in, or otherwise supported by the shaft 78b of the first stage block 78 opposite to the position sensor 64 on the third stage block 84. This position sensor 64 in conjunction with the magnet 88 on the first stage block 78 may be used to detect the relative pitch and roll movements among the first, second, and third stage blocks 78, 80, 84 as described above.

A fourth stage block 90 may be provided, which may have a planar base 90a and two attachment steps 90b at opposite sides of the planar base 90a in the Y-direction. In the example shown in FIGS. 7-10, the third stage block 84 preferably does not contact the fourth stage block 90 in a rest configuration. Rather, the third stage block 84 may be suspended using two pairs of parallel, spaced-apart third flexures 92, each having one end attached to a generally X-direction facing surface of the planar base 84a of the third stage block 84 and the other end attached to a generally X-direction facing surface of the planar base 90a of the fourth stage block 90. As before, the third flexures 92 are preferably spring plates, although other types of flexures may be used as well. In this example, each of the third flexures 92 extends in a direction generally parallel to the Z-direction. This alignment preferably places the degree of freedom of the third flexures 92 to allow for translation in the X-direction of the third stage block 84 relative to the fourth stage block 84 and uses the degrees of constraint of the third flexures 92 to prevent other types of relative motion between the third and fourth stage blocks 84, 90.

In the example shown in FIGS. 7-10, a second position sensor 64 (in this instance, a Hall effect sensor), may be attached to, embedded in, or otherwise supported by the fourth stage block 90 for movement therewith (this particular position sensor 64 is not visible in FIGS. 7-10). In this example, the second position sensor 64 may be attached to, embedded in, or otherwise supported by a Y-direction facing surface of the planar base 90a or other component of the fourth stage block 90. A magnet 88 (not visible in FIGS. 7-10) may be attached to, embedded in, or otherwise supported by a Y-direction facing surface of a component of the third stage block 84, such as a magnet arm 84c that extends away from the planar base 84a of the third stage block 84 generally parallel to the Z-direction. The second position sensor 64, in conjunction with the magnet 88 on the opposing third stage block 84, may be used to detect the relative translational movement in the X-direction of the third and fourth stage blocks 84, 90, as described above.

A fifth stage block 94 may be provided, which may have a planar base 94a, two attachment walls 94b at opposite sides of the planar base 94a in the Y-direction, and a T-bridge 94c extending from the planar base 94a in a direction generally parallel to the Z-direction. In the example shown in FIGS. 7-10, the planar base 94a of the fifth stage block 94 is located between the third and fourth stage blocks 84, 90 in the Z-direction and the T-bridge 94c passes from the planar base 94a of the fifth stage block 94 past the fourth stage block 90 in the Z-direction. However, it is preferable that the fifth stage block 94 does not contact either the third stage block 84 or the fourth stage block 90 in a rest configuration. Rather, the fourth and fifth stage blocks 90, 94 may be connected to one another using two pairs of parallel, spaced-apart fourth flexures 96, each having one end attached to a generally Y-direction facing surface of one of the attachment steps 90b of the fourth stage block 90 and the other end attached to a generally Y-direction facing surface of one of the attachment walls 94b of the fifth stage block 94. As before, the fourth flexures 96 are preferably spring plates, although other types of flexures may be used as well. In this example, each of the fourth flexures 96 extends in a direction generally parallel to the Z-direction. This alignment preferably places the degree of freedom of the fourth flexures 96 to allow for translation in the Y-direction of the fourth stage block 90 relative to the fifth stage block 94 and uses the degrees of constraint of the fourth flexures 96 to prevent other types of relative motion between the fourth and fifth stage blocks 90, 94.

A sixth stage block 98 may be provided, which may have a bulk portion 98a and a platform portion 98b extending from the bulk portion 98a in a direction generally parallel to the Z-direction. In the example shown in FIGS. 7-10, the platform portion 98b of the sixth stage block 98 passes from the bulk portion 98a past the planar base 94a of the fifth stage block 94 in the Z-direction. However, it is preferable that the sixth stage block 98 does not contact the fifth stage block 94 in a rest configuration. Rather, the fifth and sixth stage blocks 94, 98 may be connected to one another using two pairs of parallel, spaced-apart fifth flexures 100, each having one end attached to a generally Z-direction facing surface of the T-bridge 94c or the planar base 94a of the fifth stage block 94 and the other end attached to a generally Z-direction facing surface of one the platform portion 98b or the bulk portion 98a of the sixth stage block 98. As before, the fifth flexures 100 are preferably spring plates, although other types of flexures may be used as well. In this example, each of the fifth flexures 100 extends in a direction generally parallel to the Y-direction. This alignment preferably places the degree of freedom of the fifth flexures 100 to allow for translation in the Z-direction of the fifth stage block 94 relative to the sixth stage block 98 and uses the degrees of constraint of the fifth flexures 100 to prevent other types of relative motion between the fifth and sixth stage blocks 94, 98. The bulk portion 98a of the sixth stage block 98 may be secured or formed to the adjustment bracket 56, or the bulk portion 98a may be attached to the sensor enclosure 54, which in turn may be attached to the adjustment bracket 56. In any event, the sixth stage block 98 preferably serves as a fixed reference point for movements caused by the finger in the finger cup 50.

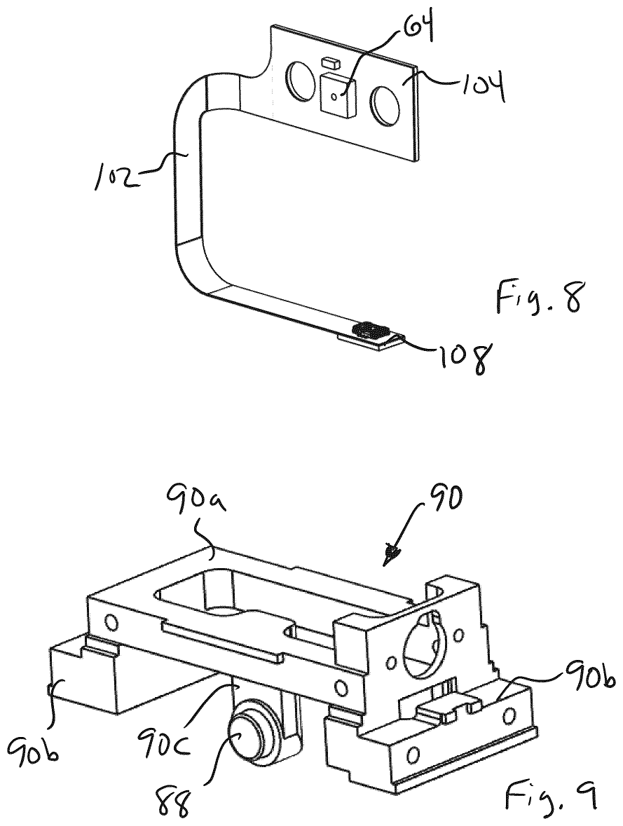

In the example shown in FIGS. 7-10, a third position sensor 64 (in this instance, a Hall effect sensor), may be attached to, embedded in, or otherwise supported by the sixth stage block 98 for movement therewith (this particular position sensor 64 can be seen in FIG. 8). In this example, the third position sensor 64 may be attached to, embedded in, or otherwise supported by an X-direction facing surface of a sensor support beam 98c or other component of the sixth stage block 98. A magnet 88 (see FIG. 9) may be attached to, embedded in, or otherwise supported by a Z-direction facing surface of a component of the fourth stage block 90, such as a magnet finger 90c that extends away from the planar base 90a of the fourth stage block 90 generally parallel to the Z-direction. The third position sensor 64, in conjunction with the corresponding magnet 88 on the fourth stage block 90, may be used to detect the relative translational movements in the Y-and Z-directions of the fourth, fifth, and sixth stage blocks 90, 94, 98, as described above.

Although the various stage blocks have been shown and described as having the particular architecture and assembled configuration presented in FIGS. 7-10, any number of stage blocks, of any desired shapes, and with any number of connecting flexures may be used, as needed by the number of degrees of freedom sought. For example, portions relevant to X-direction translation may be shaped, oriented, or configured differently from that shown. In another example, Y-and Z-direction translation are measured using one position sensor 64 and the fourth through sixth stage blocks 90, 94, 98, but these translations may be decoupled and detected by separate sensors and/or combined with other directional movements. The main aspect of the finger sensor stage assembly 52 is to isolate degrees of freedom to be measured by the position sensors 64.

Each position sensor 64 may be mounted to a flexible printed circuit 102 having an attachment face portion 104 at one end thereof. The position sensor 64 may be formed on the attachment face portion 104, which may then be secured to the appropriate stage block, such as by screws and a backer plate 106. In this manner, the position sensor 64 may be suspended across an opening, such as sensor opening 90d in the fourth stage block 90 (FIG. 9) for being placed in proximity to the corresponding magnet 88. The opposing end of the flexible printed circuit 102 may include a connecting pad 108 for coupling to a finger hub printed circuit board 110 mounted to the finger sensor stage assembly 52 or elsewhere on the finger sensor assembly 48. The flexible printed circuit 102 may enable communication from the position sensor 64 to the finger hub PCB 110 via I2C communication protocol or the like. The finger hub PCB 110 may be responsible for reporting out data received from the position sensors 64, such as via a communication port 114, as will be described in further detail below. A flex guide 112 may be provided to guide the flexible printed circuits 102 toward the finger hub PCB 110 and to prevent the flexible printed circuits 102 from coming into contact with sharp and/or moving components that could cause damage to the signal connection.

While described and shown herein using a particular configuration, the position sensors 64 and corresponding magnets 88 may be mounted in any manner desired that allows the effect of detecting isolated movements in various degrees of freedom. Similarly, the position sensors 64 may be connected to the finger hub PCB 110 or other circuitry using conventional signal cables or the like. In some embodiments, the position sensors 64 may be able to communicate wirelessly.

The finger sensor assembly 48 described above resides within the finger sensor mount 36. However, a thumb sensor assembly 49 disposed in the thumb sensor mount 38 may be similar to the finger sensor assembly 48 in many respects. That is, the thumb sensor assembly 49 may include a thumb cup 51 similar to the finger cup 50, and with a thumb cup lever 75 and associated components for clamping the user's thumb in place within the thumb cup 51, as with the finger cup lever 74 and its associated components. The thumb sensor assembly 49 may also include an adjustment lever 63 and associated components, similar to the adjustment lever 62 and its associated components, to enable the thumb sensor assembly 49 to be repositioned within the thumb sensor mount 38 to accommodate differing hand sizes. The thumb sensor assembly 49 may include a finger sensor stage assembly identical to the finger sensor stage assembly 52 found in the finger sensor assembly 48. One of the main differences shown in the drawings of the thumb sensor assembly 49 from the finger sensor assembly 48 is that the thumb cup lever 75 may be mounted generally transverse to an axis of the adjustment lever 63 on the thumb sensor assembly 49, whereas in the finger sensor assembly 48, the finger cup lever 74 is oriented generally parallel with an axis of the adjustment lever 62. This configuration is mainly to allow ease of access to the thumb cup lever 75 without interference from the user's hand or other portions of the hand therapy device 10. There may be configurations and embodiments where the thumb sensor assembly 49 and the finger sensor assembly 48 take on identical structural shapes and configurations. It is also contemplated that the thumb sensor assembly 49 can be constructed much differently from the finger sensor assembly 48, if desired.

Referring now to FIG. 11, each position sensor 64 is shown connected via its associated flex printed circuit 102 to the corresponding finger hub PCB 110 (this includes a finger hub PCB 110 for the thumb sensor assembly 49). Each finger hub PCB 110 reports data to a main board 116 of the hand therapy device 10, which may be located within the housing 34, although the main board 116 may also be located in, for example, the base 12, the arm rest 14, or the like. The connection of the finger hub PCB 110 to the main board 116 is preferably a wired connection, via the communication port 114 on the finger hub PCB 110, and may utilize the I2C protocol. As shown in FIG. 11, three separate channels are established, one for each of the position sensors 64 associated with the finger hub PCB 110. However, other types of wired protocols, as well as wireless communication, may be used as well to report the position sensor 64 data to the main board 116.

The main board 116 may include a microcontroller unit (MCU) 118 or other type of processing unit thereon, which may be configured to receive the inputs from the finger hub PCBs 110. As shown in FIG. 11, the MCU 118 may include three pins 120a, 120b, 120c for separately receiving the X, YZ, and pitch-roll data from each of the finger hub PCBs 110. However, all data could be received over one pin, or the MCU 118 could include a pin dedicated for each position sensor 64, or the like. The MCU 118 preferably takes the signals from the finger hub PCBs 110 and converts the data into force readings in the degrees of freedom under consideration. In the example shown, the position sensors 64 and the MCU 118 may have a force resolution on the order of milli-Newtons, up to approximately 7 N. The flexures in the finger sensor stage assemblies 52 are preferably configured to have appropriate stiffnesses to permit motion of the stage blocks under such small forces. The amount of motion allowed may be on the order of about 0.2 mm with an applied load of about 7 N of force or about 98 mN. m of torque, with the magnet and sensor configured to be most sensitive over a slightly larger displacement (about 0.5 mm) to capture stage motion and account for tolerances of the parts and assembly. This would allow the hand therapy device 10 to capture and analyze finger movements that would be otherwise imperceptible to visual review by a doctor or other evaluator. However, other resolutions and force measurement limits may be used as desired. In addition, the conversion of the data to force values may take place elsewhere, such as in the finger hub PCB 110 or even the flexible printed circuit 102, for example.

The main board 116 may further include a USB port 122, which may be connected to the MCU 118, to allow for an external computer (e.g., a desktop, laptop, smartphone, tablet, or the like-not shown) to connect to the hand therapy device 10. The force data calculated as described above may be reported to the external computer (such as for display of results and/or further analysis of the data) via the USB port 122, as may other data related to the hand therapy device 10 that may be of interest. In addition, the external computer may send commands to the MCU 118 for operating the hand therapy device 10, such as commands to begin or end data capture, execute programming, update firmware, or the like. While a USB port 122 is shown, other wired connections to an external computer or network may be used as well, such as an Ethernet port, IEEE 1394, or the like. In addition, the main board 116 may include one or more wireless communication modules for similar communication to an external computer, such as a BLUETOOTH module 124 and/or a WI-FI module 126. Other types of wireless communication may be used as well using various protocols, such as ZIGBEE, Z-WAVE, 3G, 4G, or 5G cellular, infrared, or the like.

In some embodiments, the MCU 118 may have the ability to conduct a zero offset to compensate position sensor 64 data due to effects of gravity. A command may be sent by the external computer via the USB port 122 or other communication interface or may be initialized by depressing one or more push buttons 128 on the hand therapy device 10 itself, for example.

The MCU 118 will then read the data from the various position sensors 64, preferably with the finger cups 50 empty, for use as a baseline while only gravity is acting on the position sensors 64 and their respective magnets 88. In other embodiments, the main board 116 may include or connect to an inertial measurement unit (IMU) 130, which may be configured to obtain a relative orientation of the hand module assembly 16 to provide data to the MCU 118 for compensating position sensor 64 readings for gravity or the like.

The hand therapy device 10 may be operated on one or more batteries 132, which may be rechargeable, either by removal from the device 10 for placement in a separate charging station (not shown) or by connecting a cable (not shown) to the hand therapy device 10 for electrical communication with the battery 132. Alternatively, the hand therapy device 10 may be powered directly from an electrical outlet or other power source (not shown).

In use, the hand therapy device 10 may first be adjusted to place the user's hand in a comfortable and neutral position. For example, the position of the hand module assembly 16 may be adjusted relative to the arm rest 14 via the wrist knob 26, the position of the chassis 42 within the housing 34 may be adjusted using the finger knob 40, the positions of the individual finger sensor assemblies 48 and the thumb sensor assembly 49 relative to the housing 34 may be adjusted using the respective adjustment levers 62, 63, and/or the finger cups 50 and thumb cup 51 may be size adjusted using the respective finger cup levers 74 and thumb cup lever 75. To ensure consistent positioning for the same user over multiple uses of the hand therapy device 10, it is preferable that each adjustable component be accompanied by an alphanumeric or otherwise human or machine readable scale indicative of positioning and/or relative orientation. For example, the indicator slot 44 may be labelled (not shown) with numerical digits representing different possible positions of the indicator pin 46 extending therein. When an appropriate position of the chassis 42 is achieved, the indicator pin 46 will align with a number on the labelled indicator slot 44. The number is preferably recorded in a patient file or elsewhere, such as in the external computer or the like, so that the next time the user utilizes the hand therapy device 10, the chassis 42 may be returned to the same position. The same is preferable for all of the adjustable components-the settings may be recorded for re-use at a later date. In some embodiments, the hand therapy device 10 may include sensors (not shown) to detect the positions of the adjustable components and may automatically send the readings to the external computer for recording.

Once the hand therapy device 10 is properly adjusted for the user's hand, the zero offset may be initiated to account for gravity in the device's set orientation. The user's hand is thereafter secured using, for example, wrist straps and the finger cup levers 74 and thumb cup lever 75. The hand therapy device 10 can thereafter begin collecting data on forces exerted by individual fingers in the applicable degrees of freedom. Data may be collected serially-for example, the user may move one finger at a time and the appropriate position sensors 64 may be active for the moving finger. The external computer and/or the MCU 118 may selectively power particular components in this operating mode. Alternatively, all position sensors 64 may be active, although the user may be instructed to move particular fingers in particular directions one at a time.

In some embodiments, a user (or an administrator, such as a doctor, therapist, or the like) may collect data using the hand therapy device 10 in an unstructured manner. For example, a doctor may simply collect data using the hand therapy device 10 for various finger maneuvers as the doctor sees fit. In some other embodiments, the external computer may execute software with one or more organized programs for collecting data. For example, the software may set a particular order for a user to perform certain movements with their fingers and for collecting the resulting data. For example, the software may instruct a user to pull their index finger toward their palm, record the resulting data, and move on to the next maneuver. Instructions could be read from the external computer by the user or aloud by the administrator, or the external computer may provide audio cues for performing each movement. In still other embodiments, the hand therapy device 10 itself may include a display (not shown) or other visual/audio indicators to instruct the user. In still other embodiments, the hand therapy device 10 may be self-contained without requirement of an external computer for completing various training and/or therapeutic operations.

Collected data also does not necessarily need to be in the form of force values. As but one example, in some embodiments, the user may be instructed to perform a task, and the position sensors 64 and MCU 118 may detect that the task has been adequately completed. The logged data may simply indicate whether the user has performed the task or not.

In addition, the hand therapy device 10 may be used not only for diagnostic purposes or for re-training a user's finger dexterity following an adverse neurologic or physical event. For example, the hand therapy device 10 may be used to train a user for playing a musical instrument, which requires finger dexterity. An appropriate training program running on the hand therapy device 10 and/or the external computer can instruct the user to manipulate their fingers in a way to train for playing, e.g., a piano or other musical instrument with their fingers. In still other embodiments, the hand therapy device 10 may be used as an input controller for a video game.

While the embodiment described herein has been generally described as having a centralized MCU 118 for performing various operations, the MCU 118 may include a plurality of individual processors or other types of controllers, with functions divided among the individual devices. In addition, some of the functionality attributed to the MCU 118 above may be distributed to other components, such as a processing device (not shown) on the finger hub PCB 110, for example.

Those skilled in the art will recognize that boundaries between above-described operations are merely illustrative. Multiple operations may be combined into a single operation, a single operation may be distributed in additional operations and operations may be executed at least partially overlapping in time. Further, alternative embodiments may include multiple instances of a particular operation, and the order of operations may be altered in various other embodiments.

Unless stated otherwise, terms such as “first” and “second” are used to arbitrarily distinguish between the elements such terms describe. Thus, these terms are not necessarily intended to indicate temporal or other prioritization of such elements. The fact that certain measures are recited in mutually different claims does not indicate that a combination of these measures cannot be used to advantage.

While specific and distinct embodiments have been shown in the drawings, various individual elements or combinations of elements from the different embodiments may be combined with one another while in keeping with the spirit and scope of the invention. Thus, an individual feature described herein only with respect to one embodiment should not be construed as being incompatible with other embodiments described herein or otherwise encompassed by the invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined herein.

We claim:

1. A finger sensor stage assembly connected to a finger cup configured to receive at least a portion of a finger, the finger sensor stage assembly being configured to detect motion in at least two degrees of freedom caused by forces generated by the finger on the finger cup, the finger sensor stage assembly comprising:
a first stage block coupled to the finger cup;
a second stage block;
at least one first flexure connected to the first stage block and to the second stage block and restricting relative movement of the first stage block and the second stage block to a first degree of freedom;
a third stage block;
at least one second flexure connected to the second stage block and to the third stage block and restricting relative movement of the second stage block and the third stage block to a second degree of freedom different from the first degree of freedom; and
one or more non-contact position sensors configured to detect the relative movement of the first stage block and the second stage block in the first degree of freedom and the relative movement of the second stage block and the third stage block in the second degree of freedom.

2. The finger sensor stage assembly of claim 1, further comprising:
a fourth stage block; and
at least one third flexure connected to the third stage block and to the fourth stage block and restricting relative movement of the third stage block and the fourth stage block to a third degree of freedom different from the first and second degrees of freedom,
wherein the one or more non-contact position sensors are further configured to detect the relative movement of the third stage block and the fourth stage block in the third degree of freedom.

3. The finger sensor stage assembly of claim 2, further comprising:
a fifth stage block; and
at least one fourth flexure connected to the fourth stage block and to the fifth stage block and restricting relative movement of the fourth stage block and the fifth stage block to a fourth degree of freedom different from the first, second, and third degrees of freedom,
wherein the one or more non-contact position sensors are further configured to detect the relative movement of the fourth stage block and the fifth stage block in the fourth degree of freedom.

4. The finger sensor stage assembly of claim 3, further comprising:
a sixth stage block; and
at least one fifth flexure connected to the fifth stage block and to the sixth stage block and restricting relative movement of the fifth stage block and the sixth stage block to a fifth degree of freedom different from the first, second, third, and fourth degrees of freedom,
wherein the one or more non-contact position sensors are further configured to detect the relative movement of the fifth stage block and the sixth stage block in the fifth degree of freedom.

5. The finger sensor stage assembly of claim 4, wherein the one or more non-contact position sensors comprise:
a first magnet supported by the first stage block and a corresponding first Hall effect sensor supported by the third stage block, the first magnet and first Hall effect sensor being configured to detect the relative movement of the first stage block and the second stage block in the first degree of freedom and the relative movement of the second stage block and the third stage block in the second degree of freedom,
a second magnet supported by the third stage block and a corresponding second Hall effect sensor supported by the fourth stage block, the second magnet and the second Hall effect sensor being configured to detect the relative movement of the third stage block and the fourth stage block in the third degree of freedom, and
a third magnet supported by the fourth stage block and a corresponding third Hall effect sensor supported by the sixth stage block, the third magnet and the third Hall effect sensor being configured to detect the relative movement of the fourth stage block and the fifth stage block in the fourth degree of freedom and the relative movement of the fifth stage block and the sixth stage block in the fifth degree of freedom.

6. The finger sensor assembly of claim 4, wherein the first and second degrees of freedom are rotational and the third, fourth, and fifth degrees of freedom are translational.

7. The finger sensor assembly of claim 1, wherein each of the at least one first flexure and the at least one second flexure is a spring plate.

8. The finger sensor assembly of claim 7, wherein the at least one first flexure comprises two pairs of parallel, spaced-apart spring plates and the at least one second flexure comprises two pairs of parallel, spaced-apart spring plates.

9. The finger sensor assembly of claim 1, wherein each of the one or more non-contact position sensors comprises a magnet and a corresponding Hall effect sensor.

10. The finger sensor assembly of claim 9, wherein the one or more non-contact position sensors comprises a single non-contact position sensor having the magnet supported by the first stage block and the Hall effect sensor supported by the third stage block.

11. The finger sensor assembly of claim 1, further comprising a finger hub printed circuit board communicatively connected to the one or more non-contact position sensors for receiving measurement data from the one or more non-contact position sensors.

12. The finger sensor assembly of claim 11, wherein the finger hub printed circuit board includes a communication port configured to transmit the measurement data received from the one or more sensors.

13. A hand therapy device comprising five finger sensor stage assemblies of claim 1.

14. A hand therapy device comprising:
a hand module assembly including:
a housing, and
five finger sensor assemblies arranged at least partially within the housing, each of the finger sensor assemblies including:
a finger cup configured to receive at least a portion of a finger,
a finger sensor stage assembly connected to the finger cup and including one or more non-contact position sensors configured to detect motion in at least two degrees of freedom caused by forces generated by the finger on the finger cup,
an adjustment slide rail, and
an adjustment bracket connected to at least one of the finger cup or the finger sensor stage assembly and selectively movable and lockable relative to the adjustment slide rail in at least one translational direction and rotatably,
wherein a position of one of the finger cups relative to the other finger cups is adjustable by movement of the corresponding adjustment bracket relative to the corresponding adjustment slide rail.

15. The hand therapy device of claim 14, further comprising a base, the hand module assembly being supported by the base.

16. The hand therapy device of claim 15, further comprising an arm connecting the base to the hand module assembly.

17. The hand therapy device of claim 16, wherein the arm is rotatable with respect to the base.

18. The hand therapy device of claim 17, further comprising:
a rotating collar supported by the base and connected to the arm; and
a wrist knob attached to the rotating collar, the wrist knob including a rotatable dial such that rotating the rotatable dial in a first direction expands the rotating collar to allow the rotating collar to rotate relative to the base and adjust a position of the arm relative to the base, and rotating the rotatable dial in a second direction opposite to the first direction tightens the rotating collar to prevent rotation of the rotating collar relative to the base and lock the arm in position relative to the base.

19. The hand therapy device of claim 15, further comprising an arm rest supported by the base.

20. The hand therapy device of claim 14, wherein the hand module assembly further includes a chassis selectively movable and lockable relative to the housing, the chassis containing at least four of the finger sensor assemblies.

21. The hand therapy device of claim 20, wherein the housing includes an indicator slot and the chassis includes a cooperating indicator pin extending within the indicator slot, the indicator pin allowing selective translational movement of the chassis relative to the housing in a direction parallel to a longitudinal direction of the indicator slot.

22. The hand therapy device of claim 21, wherein the hand module assembly further includes a finger knob rotatably mounted on the housing, wherein rotating the finger knob in a first direction allows the chassis to move relative to the housing as guided by the indicator pin within the indicator slot, and rotating the finger knob in a second, opposite direction locks the chassis to the housing.

23. The hand therapy device of claim 14, wherein the hand module assembly further includes a microcontroller unit disposed within the housing and communicatively connected to each of the one or more sensors from the five finger sensor assemblies.

24. The hand therapy device of claim 23, wherein the microcontroller unit includes at least one communication module for sending data related to the one or more sensors.

25. The hand therapy device of claim 24, wherein the at least one communication module includes a USB port.

26. The hand therapy device of claim 14, wherein each of the finger sensor assemblies further includes:
a brake housing attached to the adjustment bracket and selectively movable with respect to the adjustment slide rail, and
an adjustment lever selectively pivotable relative to the brake housing, wherein when the adjustment lever is in a first position relative to the brake housing, the brake housing and the adjustment bracket are movable relative to the adjustment slide rail, and when the adjustment lever is in a second position relative to the brake housing, the brake housing and the adjustment bracket are prevented from moving relative to the adjustment slide rail.

27. The hand therapy device of claim 26, wherein the adjustment lever is biased toward the second position by a spring plunger positioned between the brake housing and the adjustment lever.

28. The hand therapy device of claim 14, wherein each finger cup includes a finger cup base and a finger cup top that is selectively movable toward or away from the finger cup base.

29. The hand therapy device of claim 28, wherein each finger cup further includes a finger cup lever and a lever support, the lever support being connected to the finger cup top for movement therewith, the finger cup lever being selectively pivotable with respect to the lever support,
wherein when the finger cup lever is in a first position relative to the lever support, the lever support and finger cup top are free to move relative to the finger cup base, and when the finger cup lever is in a second position relative to the lever support, the lever support and finger cup top are prevented from moving relative to the finger cup base.

30. A method of operating a hand therapy device, the hand therapy device including a microcontroller unit and a hand module assembly that includes a housing and five finger sensor assemblies arranged at least partially within the housing, each of the finger sensor assemblies including a finger cup configured to receive at least a portion of a finger and a finger sensor stage assembly connected to the finger cup and including one or more non-contact position sensors configured to detect motion in at least two degrees of freedom caused by forces generated by the finger on the finger cup, the method comprising:
adjusting a position of at least one of the finger sensor assemblies relative to the other finger sensor assemblies to establish a neutral rest position for a hand of a user to be received by the hand module assembly;
recording the positions of the finger sensor assemblies after establishing the neutral rest position for the hand of the user;
receiving, by the finger cups of the finger sensor assemblies, corresponding fingers of the hand of the user; and
obtaining, by the microcontroller unit, data from the one or more non-contact position sensors of at least one of the finger stage assemblies, the data representing movement of the respective at least one finger stage assembly caused by forces generated by the respective finger.

31. The method of claim 30, further comprising:
resetting, by the microcontroller unit after establishing the neutral rest position for the hand of the user, the one or more non-contact position sensors in the finger stage assemblies to account for a direction of gravity relative to the finger stage assemblies.

32. The method of claim 30, wherein the hand therapy device further includes an arm rest and a base supporting the arm rest and the hand module assembly, the method further comprising:
adjusting a position of the hand module assembly relative to the arm rest to establish the neutral rest position for the hand of the user; and
recording the position of the hand module assembly after establishing the neutral rest position for the hand of the user.

33. The method of claim 30, wherein a size of the finger cups of the finger sensor assemblies is adjustable, the method further comprising:
adjusting the size of at least one of the finger cups; and
recording the sizes of the finger cups after establishing the neutral rest position for the hand of the user.

34. The method of claim 30, wherein the hand module assembly further includes a chassis containing at least four of the finger sensor assemblies, the method further comprising:
adjusting a position of the chassis relative to the housing to establish the neutral rest position for the hand of the user; and
recording the position of the chassis after establishing the neutral rest position for the hand of the user.
